(19) United States
(12) Patent Application Publication
(10) Pub. No.: US 2010/0112643 A1
(43) Pub. Date: May 6, 2010

(54) METHOD FOR DIRECT CAPTURE OF RIBONUCLEIC ACID

(75) Inventors: Marie J. Archer, Alexandria, VA (US); Baochuan Lin, Bethesda, MD (US); David A. Stenger, Herndon, VA (US)

Correspondence Address: NAVAL RESEARCH LABORATORY;ASSOCIATE COUNSEL (PATENTS), CODE 1008.2, 4555 OVERLOOK AVENUE, S.W., WASHINGTON, DC, 20375-5320, US

(73) Assignee: The Government of the United States of America, as represented by the Secretary of the Navy, Washington, DC (US)

(21) Appl. No.: 12/264,462

(22) Filed: Nov. 4, 2008

(51) Int. Cl. C12P 19/34; C07H 21/02

(52) U.S. Cl. 435/91.2; 536/24.3

(57) ABSTRACT

A method of: providing a solid surface having a dendrimer molecule bound thereto and a single-stranded probe nucleic acid immobilized to the dendrimer; contacting the solid surface with a sample suspected or known to contain a target ribonucleic acid; denaturing the target ribonucleic acid; and incubating the sample to allow hybridization of the denatured ribonucleic acid to the probe nucleic acids. The target ribonucleic acid is complementary to the probe nucleic acid.

### TECHNICAL FIELD

The invention is generally related to the capture of ribonuclcic acid targets.

### DESCRIPTION OF RELATED ART

The isolation of RNA from complex matrices is required in many applications such as genomic sequencing, clinical diagnosis, environmental monitoring and even agriculture. Particularly, the identification of ribonucleic acid (RNA) viral pathogens in clinical samples represents a challenge since the abundance of RNA depends on the pathogens and the stage of the infection (Muir et al., Rapid Diagnosis of Enterovirus Infection by Magnetic Bead Extraction and Polymerase Chain Reaction Detection of Enterovirus RNA in Clinical Specimens. J. Clin. Microbiol. 31 (1993) 31-38). (All publications and patent documents referenced throughout this application are incorporated herein by reference.)

One approach to identify RNA viral pathogens is a molecular diagnostic which requires RNA extraction and performing pathogen specific reverse transcriptase-polymerase chain reaction (RT-PCR). However, the use of RT-PCR for routine analysis of viral pathogens has been limited given the labor intensive extraction step required to obtain high quality RNA with no contamination from other nucleic acids or the chemicals used in the extraction step. The process comprises a lysis step and a step to trap the hydrophobic components of the lysate (proteins and lipids) leaving the nucleic acids, salts and sugars in a liquid phase. The use of chloroform at low pH allows the separation of RNA from DNA and proteins. The drawback of this approach is the time required for the process which can be more than three hours. Also, the error associated with manual manipulation is a significant factor. Furthermore, it involves the use of highly toxic materials that can be carried to the downstream process and even in small amounts can inhibit the RT and polymerase chain reactions (PCR) (van Doom et al., Hepatitis C virus antibody detection by a line immunoassay and (near) full length genomic RNA detection and new RNA-capture polymerase chain reaction. J. Med. Virol. 38 (1992) 298-304; van Doom et al., Rapid detection of Hepatitis C virus by direct capture form blood. J. Med. Virol. 42 (1994) 22-28; Hsuih et al., Novel, ligation-dependent PCR assay for detection of hepatitis C virus in serum. J. Clin. Microbiol. 34 (1996) 501-507; Beaulieux et al., Use of magnetic beads versus guanidium thiocyanate-phenol-chloroform RNA extraction followed by polymerase chain reaction for the rapid, sensitive detection of enterovirus RNA. Res. Virol. 148 (1997) 11-15; O'Meara et al., Cooperative oligonucleotides mediating direct capture of hepatitis C Virus RNA from serum. J. Clin. Microbiol. 36 (1998) 2454-2459). The need to isolate RNA in a timely manner without the use of toxic chemicals, while preserving the integrity of the RNA has led to the development of alternate isolation methods, e.g. paramagnetic beads with a nucleic acid binding surface and silica-gel membrane columns.

Two approaches of isolating RNA using magnetic microbeads have been documented. In the single-step or direct capture approach, the target RNA is captured by a probe attached to the magnetic beads through affinity (avidin-biotin) (van Doorn et al., J. Med. Virol. 38 (1992) 298-304; Muir et al., J. Clin. Microbiol. 31 (1993) 31-38; Beaulicux et al., Res. Virol. 148 (1997) 11-15) or covalent (amido, amino, or hydroxy) bonds (Spottke et al., Reverse Sanger sequencing of RNA by MALDI-TOF mass spectrometry after solid phase purification. Nucleic Acids Res. 32 (2004) e97; Albretsen et al., Applications of magnetic beads with covalently attached oligonucleotides in hybridization: isolation and detection of specific measles virus mRNA from crude cell lysate. Anal. Biochem. 189 (1990) 40-50). The covalent attachment can be done by direct synthesis of the probe on the bead (Albretsen) or by reaction between carboxyl-, amino-, or hydroxyl- group to an end-modified capture probes (U.S. Pat. No. 5,512,439). In the two-step capture the target RNA is first hybridized in solution with a biotinylated capture probe and the captured probe-target complex is then hybridized to the streptavidin coated beads (van Doom et al., J. Med. Virol. 42 (1994) 22-28; Hsuih et al., J. Clin. Microbiol. 34 (1996) 501-507; Chandler et al., Affinity purification of DNA and RNA from environmental samples with peptide nucleic acid clamps. Appl. Environ. Microbiol. 66 (2003) 3438-3445).

The concept of “oligonucleotide-assisted capture assay” was developed which combines solution phase hybridization of the targets with a pre-hybridization probe followed by solid phase capture on streptavidin beads functionalized with a biotinylated capture probe (O'Meara et al., J. Clin. Microbiol. 36 (1998) 2454-2459; Hei et al., Development of a method for concentrating and purifying SARS coronavirus RNA by a magnetic bead capture system. DNA and Cell Biology 24 (2005) 479-484). This approach has demonstrated enhanced detection sensitivity at the cost of increasing the complexity of the assay.

A solid phase was developed for the selective capture of genomic DNA in a single step using a solid phase fabricated with a substrate functionalized with a primary amine onto which a branched phosphorus dendrimer is covalently attached (Archer et al., Magnetic bead-based solid phase for selective extraction of genomic DNA. Anal. Biochem. 355 (2006) 285-297; U.S. patent application Ser. No. 11/751,096). The suitability of this approach was demonstrated for the selective extraction of genomic DNA for background subtraction and sequence-capture applications.

### BRIEF SUMMARY

A method comprising: providing a solid surface having a dendrimer molecule bound thereto and a single-stranded probe nucleic acid immobilized to the dendrimer; contacting the solid surface with a sample suspected or known to contain a target ribonucleic acid; denaturing the target ribonucleic acid at thermal conditions sufficient to denature the target ribonucleic acids to produce denatured ribonucleic acids; and incubating the sample to allow hybridization of the denatured ribonucleic acid to the probe nucleic acids. The target ribonucleic acid is complementary to the probe nucleic acid.

### BRIEF DESCRIPTION OF THE DRAWINGS

A more complete appreciation of the invention will be readily obtained by reference to the following Description of the Example Embodiments and the accompanying drawings.

FIG. 1(a) shows a representative image of the RT-PCR products from the supernatants recovered after subtraction of 18S and 28S ribosomal RNA using magnetic microbeads functionalized with a generation 4.5 dendrimer. Lane 1, molecular weight marker; lane 2, negative control; lane 3, positive control for 18S ribosomal RNA (250 ng of total RNA); lane 4, positive control for 18S ribosomal RNA (100 ng of total RNA); lane 5, product after subtraction; lanes 6 and 7, reference samples (no subtraction); lane 8, product of a blank target. FIG. 1(b) shows a representative image of the RT-PCR products of Influenza A/H1N1 from the supernatants recovered after subtraction of 18S and 28S ribosomal RNA. Lane 1, molecular weight marker; lane 2, negative control; lane 3, positive control (1 ng of Influenza A/H1N1); lane 4, reference sample (no subtraction); lane 5, product after subtraction.

FIG. 2(a) shows a representative image of the RT-PCR products from the enriched influenza A/H1N1 using magnetic microbeads functionalized with a generation 4.5 dendrimer. Lane 1, molecular weight marker; lane 2, negative control; lane 3, positive control (1 ng of influenza A/H1N1); lane 4, supernatant of a blank target; lane 5, supernatant of a reference sample; lane 6, supernatant after capture; lane 7, eluted product of a blank target; lane 8, eluted product from the beads used to capture Influenza A H1N1. FIG. 2(b) shows a representative image of the RT-PCR products of the 18S gene of the ribosomal RNA from the supernatants recovered after capture of Influenza A/H1N1. Lane 1, molecular weight marker; lane 2, negative control; lane 3, positive control (250 ng of total RNA); lane 4, product of a blank target; lane 5, reference sample (no capture)and lane 5, product after capture.

FIG. 3 shows a representative image of the RT-PCR products from the enriched influenza A/H1N1 RNA using magnetic microbeads functionalized with a generation 4.5 phosphorous dendrimer. Lane 1, molecular weight marker; lane 2, negative control; lane 3, positive control (1 ng of influenza A/H1H1 RNA); lane 4, eluted product of a blank target; and lane 5, eluted product of influenza A/H1N1 RNA.

FIG. 4 shows a representative image of the supernatants recovered after subtraction of 18S and 28S ribosomal RNA. Lane 1, molecular weight marker; lane 2, control sample (no subtraction; lane 3, supernatant after capture with magnetic microbeads functionalized with a generation 0.5 dendrimer; lane 4, supernatant after capture with magnetic microbeads functionalized with a generation 4.5 dendrimer. The band corresponding to the 18S and 28S rRNA are marked on the reference lane (Lane 1)

### DETAILED DESCRIPTION OF EXAMPLE EMBODIMENTS

In the following description, for purposes of explanation and not limitation, specific details are set forth in order to provide a thorough understanding of the present disclosure. However, it will be apparent to one skilled in the art that the present subject matter may be practiced in other embodiments that depart from these specific details. In other instances, detailed descriptions of well-known methods and devices are omitted so as to not obscure the present disclosure with unnecessary detail.

The process described herein provides a method for selective capture of RNA from complex matrices. This disclosure presents a general method for the selective capture of RNA that can be used for either target enrichment or background subtraction applications. Additionally, this method can be adapted for simultaneous target enrichment and background subtraction by using different solid phases, i.e. different size magnetic beads, or magnetic beads with glass beads. The method described herein utilizes the magnetic bead-based solid phase functionalized with a phosphorous dendrimer linker disclosed in U.S. patent application Ser. No. 11/751,096 that allows control of the performance by changing the probe loading capacity. The thermal stability of the solid phase allows the method to be performed in a single-step process and recovery of the targets to be performed through heat denaturation which greatly reduced the processing steps involved in performing the experiments.

This invention enables the selective capture of genomic RNA to be performed in a single step in which the RNA is first denatured at, for example, about 70° C. to about 80° C., and then captured by the probes immobilized on the solid phase. The denaturing may be performed under any conditions, including temperature and the use of RNA binding buffer, that results in the denaturing. The denatured RNA is then incubated with the solid support and probes at any temperature that allows hybridization of the RNA to the probes, for example about 40° C. to about 50° C. The captured targets can be released (eluted) from the solid phase through heat denaturation at, for example, at about 70 to about 80° C., and the solid phase used for a new capture.

The substrate material can be, but is not limited to, structured silicon, solid glass microbeads, silica beads, planar silicon wafer, and paramagnetic microbeads. Disclosed is a particular process for preparing the microparticles for the covalent immobilization of phosphorous dendrimers. The silicon and glass solid supports can be prepared by the methods described by US Patent Application Publication 2005/0214767.

The selectivity of the solid phase is conferred by immobilizing probes onto the functionalized solid supports. This is performed by exposing the solid support to a solution containing amino modified double stranded DNA capture probes with a length between 200 and 800 bp. A covalent bond is produced between the amino terminal of the DNA capture probe and the aldehyde reactive functions of the dendrimer. Suitable probes include, but are not limited to, single-stranded DNA, single-stranded human DNA, and single-stranded pathogen DNA. The probes are single-stranded when attached to the dendrimer, but may have been derived from double-stranded sources.

The “bowl-shaped” dendrimers differ in size and in number of branches available for covalent coupling with amines. The aldehyde groups (—CHO) on the periphery of the dendrimer are electrically neutral, reducing the electrostatic interactions with the DNA capture probes and facilitating the covalent attachment through the aminated end. Also, the use of phosphorus dendrimers as the linker system provides the high loading capacity, reduced steric hindrance, and thermal stability required for the fabrication of a selective solid phase. In the following, the term G4.5 or G0.5 SiO₂ section refers to a section of silicon oxide wafer (4×7 mm) functionalized with G4.5 or G0.5 phosphorus dendrimer. Likewise, the term G4.5 or G0.5 magnetic beads refers to magnetic beads functionalized with a G4.5 or G0.5 phosphorus dendrimer. The structure of a suitable dendrimer is shown below. The value n is a nonnegative integer, but need not be the same in each branch. The term “G4.5” refers to a dendrimer where n is 4 and X is aldehyde or other terminating group (as opposed to an intermediate chloride). Note that when n is 0, there are a total of 6-O—C₆H₆—X groups in the compound (N₃P₃(OC₆H₆X)₆). N₃P₃ is hexavalent cyclotriphosphazene. Each X is independently selected from —CHO and —CH₂—NH—. Each —CH₂—NH— group is directly or indirectly bound to the solid surface or the probe nucleic acid. There is at least one —CH₂—NH— group directly or indirectly bound to the solid surface and at least one —CH₂—NH— group directly or indirectly bound to the probe nucleic acid. The repeating unit in parentheses indicates that phosphorous atom on the right is bound to two repeat units.

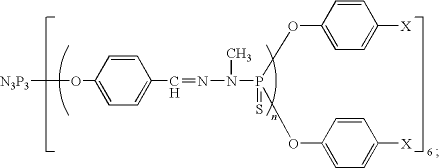

Several additional steps may be performed. The solid surface may be separated from the sample. In this step and in other steps, the solid surface may include the entire solid phase with the dendrimer, capture probe, and optionally any captured ribonucleic acid. The remaining nucleic acids in the sample after this separation may be analyzed to identify the organism of the nucleic acids. A reverse transcription and polymerase chain reaction (RT-PCR) may be performed on the remaining nucleic acids. The hybridized nucleic acids may be removed from the solid phase after separation from the sample and a RT-PCR performed on them.

In comparison with the commercially available “custom-coupled magnetic beads” (INVITROGEN®) the disclosed method allows the fabrication of the solid phase to be done by the end user in the amounts and with the characteristics required regardless of the probe length or the number of times that a particular probe would be used. Furthermore, with the proposed method the capture probes immobilized on the solid phase can originate from double stranded PCR products and it is not restricted to single stranded short oligonucleotides. The method reduces manual handling steps which in turns decrease the chances of contamination and samples mishandling due to human error. In addition, this process may be more amenable to an automation process. An additional advantage of the method may be that background subtraction and enrichment could be performed simultaneously by immobilizing capture probes for enrichment on one bead size and capture probes for background subtraction on a different bead size. After the selective capture using a mixture of the beads, separation of each bead type based on size can be performed by magnetophoresis which is also amenable for automation (Pamme et al., On-Chip Free-Flow Magnetophoresis: Continuous Flow Separation of Magnetic Particles and Agglomerates. Anal. Chem. 76 (2004) 7250-7256). Alternatively, capture probes for enrichment and background subtraction can be immobilized on a different solid phase to achieve the same goal.

The following examples are given to illustrate specific applications. These specific examples are not intended to limit the scope of the disclosure in this application.

### EXAMPLE 1

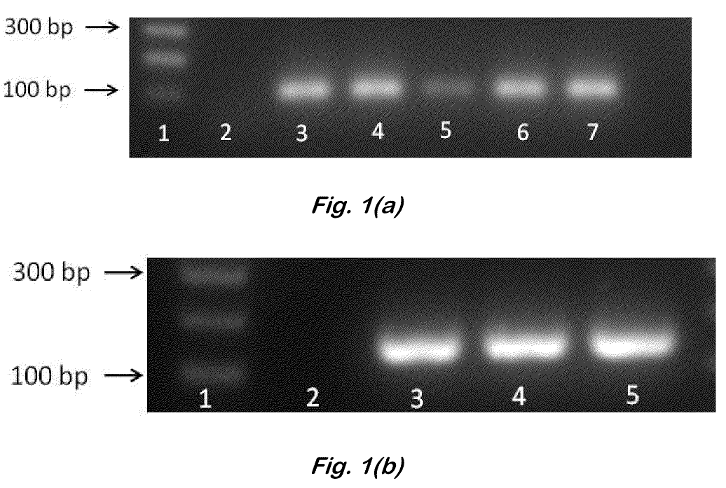

Method for subtracting human ribosomal RNA from total nucleic acids in a single step using a magnetic bead based solid phase functionalized with a generation 4.5 phosphorous dendrimer—Magnetic microbeads (˜1 μm) were functionalized with a generation 4.5 dendrimer. Capture probes for 18S and 28S ribosomal RNA (rRNA) were prepared by PCR and immobilized on the beads as described in Archer et al., Magnetic bead-based solid phase for selective extraction of genomic DNA. Anal. Biochem. 355 (2006) 285-297. The beads were then blocked for non-specific binding and the double stranded capture probes converted into single strands by heat denaturation as described in Archer. Prior to the capture experiments the beads were washed once with 100 μL of nuclease free water. The beads were mixed with 50 μL of total nucleic acids extracted from throat swabs containing human DNA (hDNA) and ribosomal RNA (rRNA), 1 ng of Influenza A/H1N1 RNA, 40 U of recombinant ribonuclease inhibitor, 100 μL of RNA binding buffer (3.8 M TMAC/0.15% SDS) and bovine serum albumin (BSA) at a concentration of 0.125 μg/μL. The concentration of the total nucleic acids used in these experiments was 25.5 ng/μL. The capture was carried out in a thermal mixer by incubating the beads with an initial denaturing step at 75° C. for 20 minutes followed by an annealing step at 50° C. for 80 minutes with continuous mixing at 1400 rpm. During the denaturing step the beads were mixed four times at 1400 rpm for 5 seconds. After capture, the supernatants from the capture were transferred to a collection tube and the beads were washed once with 100 μμL of 2×SCC/0.1% SDS solution (Wash 1) and once with 100 μL of 0.1×SSC/0.1% SDS (Wash 2). The supernatants from Wash 1 and Wash 2 were transferred to the collection tube. The recovered supernatants were precipitated and analyzed through gel electrophoresis after performing RT-PCR. The lack of probe shedding was addressed with a blank experiment using beads subject to the same process without adding the total nucleic acids. Capture of the human ribosomal RNA was addressed by RT-PCR on the 18S gene of human ribosomal RNA. Likewise, lack of non-specific capture was determined by RT-PCR on the matrix gene of influenza A/H1N1 RNA. The results are shown in FIGS. 1(a) and (b) respectively.

In FIG. 1(a), the lower intensity band on lane 5 in comparison with the references (lanes 6 and 7, no subtraction) indicates capture of rRNA. In FIG. 1(b) the presence of a band on lane 5 comparable to the reference sample (lane 4) indicates lack of non-specific capture, that is, the Influenza A/H1N1 RNA remains in the supernatant and only the ribosomal RNA is subtracted.

### EXAMPLE 2

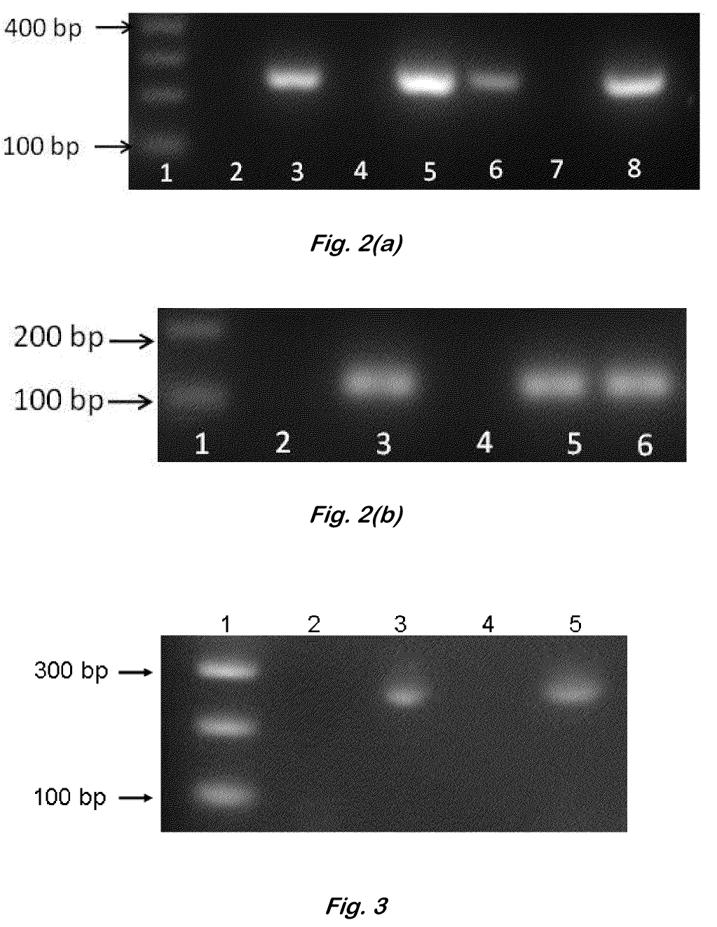

Method for enrichment of influenza A/H1N1 RNA in a single step using a magnetic bead based solid phase functionalized with a generation 4.5 phosphorous dendrimer—Magnetic microbeads (˜1 μm) were functionalized with a generation 4.5 dendrimer. Capture probes for influenza A/H1N1 were prepared using Sequenase DNA polymerase. The magnetic bead based solid phase preparation and the selective capture of influenza A/H1N1 was performed as described in Example 1 using 50 μL of total nucleic acids extracted from throat swabs at concentration of 18 ng/μL and 1 ng of Influenza A/H1N1 as the target. Recovery (elution) of the captured targets from the beads was performed by heat denaturation. For this purpose, prior to the elution of the targets, the beads were washed once with 100 μL of 0.1×SSC/0.1% Tween 20 and re-suspended in 22 μL of the same buffer. Heat denaturation of the targets was performed for 10 minutes at 72° C., the supernatant was collected and the beads were again re-suspended in 22 μL of the same buffer (0.1×SSC/0.1% Tween 20) and incubated at the same temperature for additional 10 minutes. The recovered targets were analyzed through gel electrophoresis after performing RT-PCR on half of the eluted volume. The lack of probe shedding was addressed with a blank experiment using beads with capture probes for influenza A/H1N1 subject to the same process without adding any target. Likewise, lack of non specific capture was determined by RT-PCR on the 18S gene of human ribosomal RNA. The results are shown in FIGS. 2(a) and (b) respectively.

In FIG. 2(a) the lower intensity band of lane 5 with respect to the reference indicates that most of the Influenza A/H1N1 was captured by the beads and this is further confirmed by the product obtained from the elution of the beads (lane 8). The lack of probe shedding during the capture and the elution steps is confirmed by the absence of a band on lanes 4 and 7. In FIG. 2(b) the presence of a band on lane 4 indicates that the ribosomal RNA remains in the supernatant and was not captured by the beads. Altogether these results demonstrate the enrichment of influenza A/H1N1 through selective capture followed elution (recovery) of the target form the beads.

### EXAMPLE 3

Method for enrichment of influenza A/H1N1 RNA in a single step using magnetic bead based solid phase functionalized with a generation 4.5 phosphorous dendrimer—Magnetic microbeads (˜2 μm) were functionalized with a generation 4.5 dendrimer. Capture probes for influenza A/H1N1 were prepared using Sequenase DNA polymerase and immobilized on the beads as described in Archer et al., Anal. Biochem. 355 (2006) 285-297. The beads were then blocked for non-specific binding and the double stranded capture probes converted into single strands by heat denaturation. Prior to the capture experiments the beads were washed once with 100 μL of nuclease free water and 100 μL of RNA binding buffer (250 mM Tris-HCl, 375 mM KCl, 15 mM MgCl₂) at pH 8.3. The beads were the mixed with 30 μL of nuclease free water, 28 μL of RNA binding buffer, 40 U of recombinant ribonuclease inhibitor and 1 ng of influenza A/H1N1 RNA. The capture was carried out by incubating the beads with an initial denaturing step at 70° C. for 10 minutes followed by 50° C. for 30 minutes and 37° C. for 10 minutes with ramping at 0.1° C./min. The beads were re-suspended at the beginning of the 50° C. step. Alter capture the beads were washed once with 60 μL of the RNA binding buffer, once with a 1:10 dilution of the RNA hybridization buffer (Wash 1) and once with a 1:10 dilution of Wash 1. Prior to elution the beads were washed once with 100 μL of 10 mM Tris-HCl buffer (pH 8.0) and re-suspended in 22 μL of the same buffer. Heat denaturation of the targets was performed for 10 minutes at 70° C., the supernatant was collected and the beads were again re-suspended in 22 μL of 10 mM Tris-HCl buffer (pH 8.0) and incubated at the same temperature for additional 10 minutes. The recovered targets were analyzed through gel electrophoresis after performing RT-PCR on half of the eluted volume. The lack of probe shedding was addressed with a blank experiment using beads subject to the same process without adding the target influenza A/H1N1 RNA. FIG. 3 shows a representative image of the RT-PCR products from the enriched influenza A/H1N1 RNA using magnetic microbeads functionalized with a generation 4.5 phosphorous dendrimer. Lane 1, molecular weight marker; lane 2, negative control; lane 3, positive control (1 ng of influenza A/H1H1 RNA); lane 4, eluted product of a blank target; and lane 5, eluted product of influenza A/H1N1 RNA.

### EXAMPLE 4

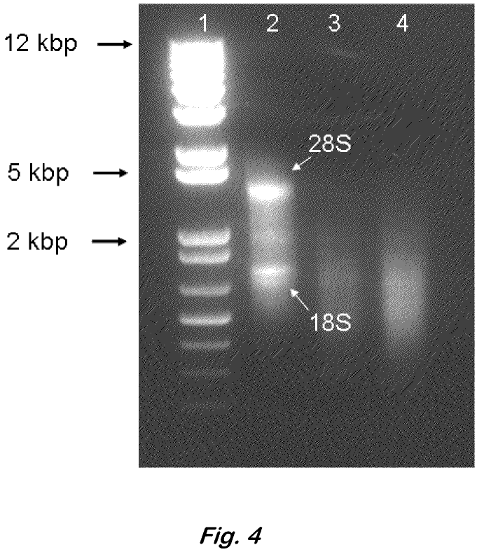

Method for subtracting 18S and 28S ribosomal RNA from total RNA using magnetic microbeads functionalized with a generation 0.5 and 4.5 phosphorous dendrimer—Magnetic microbeads were functionalized with either a generation 0.5 or a generation 4.5 phosphorous dendrimer. Capture probes for 18S and 28S ribosomal RNA (rRNA) were prepared by PCR. The solid phase preparation and the selective capture of 18S and 28S rRNA were carried out as described in Example 3. In this case no elution was performed; the supernatants after capture were precipitated for further analysis. For these experiments the target used was 1 μg of total RNA which is within the range encountered in some clinical samples. The capture efficiency was evaluated quantitatively through quantification of the remnant (unbound) RNA and qualitatively by corroborating the reduction of the 18S and 28S characteristic bands in an ethidium bromide-stained agarose gel. A reference sample (no capture) was included in the experiments. The results presented in FIG. 4 show that, for this particular application (subtraction of 18S and 28S rRNA), generation 0.5 dendrimer functionalized solid phase indicated higher subtraction efficiency. The quantitative results show a significant reduction in the total amount to RNA captured with respect to the reference sample (67 ng/μL vs 1071 ng/μL). Although the G4.5 beads also subtracted a significant amount of both targets (109 ng/μL vs 1071 ng/μL), the G4.5 beads is less efficient in subtraction the 28S rRNA (FIG. 4, lane 4). These results demonstrate that the method described here can be used to control the performance of the solid phase through the linker generation for a particular application and that the method is suitable for background subtraction applications.

### EXAMPLE 5

Method for enrichment of influenza A/H1N1 RNA from a complex matrix containing a 2000 excess fold background material—In order to demonstrate the feasibility of the method to enrich influenza A/H1N1 RNA from a matrix containing human genomic DNA (hgDNA) and total RNA (tRNA) the magnetic bead-based solid phase was functionalized with a generation 4.5 dendrimer and capture probes for influenza A/H1N1. The capture was performed as described in Example 3. Prior to the elution, the beads were washed once with 100 μL of a low salt buffer (0.1% Tween 20/0.1×SCC) re-suspended in 22 μL of the same buffer and incubated for 20 minutes at 70° C. The supernatant was recovered and the beads were re-suspended in 22 μL of pre-warmed nuclease free water at 70° C. and incubated for additional 20 minutes. The supernatant was pooled with the first recovery or a total elution volume of ˜44 μL.

For these experiments 2 ng of FluA H1N1 were spiked into 1 μg of hgDNA and 1 μg of tRNA and the enrichment was carried out as described in Example 3. The elutants were analyzed through RT-PCR, and quantified through UV/Vis spectrophotometry. A blank experiment in which beads with no target were subject to the same process was included to corroborate the lack of probe shedding. Additionally, in order to compare the enrichment efficiency with and without the presence of background a target without background material was included in the experiments. Quantification of the purified products showed 64% recovery in the presence of background material and 75% recovery without background material. These results demonstrate that the method is suitable for enrichment of low abundance RNA targets in the presence of excess background with yields comparable to those obtained when no background is present.

Obviously, many modifications and variations are possible in light of the above teachings. It is therefore to be understood that the claimed subject matter may be practiced otherwise than as specifically described. Any reference to claim elements in the singular, e.g., using the articles “a,” “an,” “the,” or “said” is not construed as limiting the element to the singular.

1. A method comprising:
providing a solid surface having a dendrimer molecule bound thereto and a single-stranded probe nucleic acid immobilized to the dendrimer;
contacting the solid surface with a sample suspected or known to contain a target ribonucleic acid;
wherein the target ribonucleic acid is complementary to the probe nucleic acid;
denaturing the target ribonucleic acid at thermal conditions sufficient to denature the target ribonucleic acids to produce denatured ribonucleic acids; and
incubating the sample to allow hybridization of the denatured ribonucleic acid to the probe nucleic acids.

2. The method of claim 1, wherein the denaturing is performed at about 70 to about 80° C.

3. The method of claim 1, wherein the denaturing is performed in an RNA binding buffer.

4. The method of claim 1, wherein incubating the sample comprises cooling the sample to about 40 to about 50° C.

5. The method of claim 1, wherein the dendrimer is: 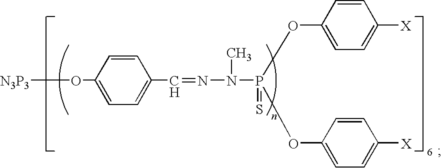
wherein each n is a nonnegative integer;
wherein N₃P₃ is hexavalent cyclotriphosphazene; and
wherein each X is independently selected from —CHO and —CH₂—NH—;
wherein each —CH₂—NH— group is directly or indirectly bound to the solid surface or the probe nucleic acid; and
wherein there is at least one —CH₂—NH— group directly or indirectly bound to the solid surface and at least one —CH₂—NH— group directly or indirectly bound to the probe nucleic acid.

6. The method of claim 1, further comprising:
separating the solid surface from the sample and any remaining nucleic acids in the sample.

7. The method of claim 6, further comprising:
analyzing the remaining nucleic acids in the sample after the sample is separated from the solid surface to identify an organism.

8. The method of claim 6, further comprising:
performing a reverse transcription and a polymerase chain reaction on the remaining nucleic acids in the sample after the sample is separated from the solid surface.

9. The method of claim 6, further comprising:
removing the hybridized ribonucleic acids from the solid surface by heat denaturation at about 70 to about 80° C. after the sample is separated from the solid surface.

10. The method of claim 9, further comprising:
performing a reverse transcription and a polymerase chain reaction on the removed ribonucleic acids.

11. The method of claim 1, wherein the surface comprises a plurality of paramagnetic microbeads.

12. The method of claim 1, wherein the surface comprises a glass slide, a silicon wafer, a structured silicon surface, a plurality of solid glass microbeads, or a plurality of silica beads.

13. The method of claim 1, wherein the sample contains human ribonucleic acids and is suspected of containing pathogen ribonucleic acids.

14. The method of claim 13, wherein the probe nucleic acids are complementary to the human ribonucleic acids.

15. The method of claim 13, wherein the probe nucleic acids are complementary to the pathogen ribonucleic acids.

16. The method of claim 14, wherein the probe nucleic acids include probes complementary to the 18S and 28S human ribosomal ribonucleic acid.
